(19) United States
(12) Patent Application Publication
(10) Pub. No.: US 2005/0019208 A1
(43) Pub. Date: Jan. 27, 2005

(54) Process for pasteurizing an oxygen sensitive product and triggering an oxygen scavenger, and the resulting package

(76) Inventors: Drew V. Speer, Simpsonville, SC (US); Thomas D. Kennedy, Simpsonville, SC (US); Ronald L. Cotterman, Greenville, SC (US)

Correspondence Address: CRYOVAC, INC.;SEALED AIR CORP, P.O. BOX 464, DUNCAN, SC, 29334, US

(21) Appl. No.: 10/917,614

(22) Filed: Aug. 13, 2004

Related U.S. Application Data
(62) Division of application No. 09/860,390, filed on May 18, 2001.

(51) Int. Cl. A61L 2/08

(52) U.S. Cl. 422/22; 422/40

(57) ABSTRACT

A package includes a container, the container including an oxygen scavenger; and an oxygen sensitive product contained in the container; wherein the oxygen scavenger is triggered, and the oxygen sensitive product is pasteurized. A package includes a tray; a lidstock in communication with the tray; and an oxygen sensitive product contained in the tray, and enclosed by the lidstock and the tray; wherein at least one of the lidstock and the tray includes an oxygen scavenger, the oxygen scavenger is triggered, and the oxygen sensitive product is pasteurized. A method includes providing a container containing an oxygen sensitive product, the container including an oxygen scavenger; and exposing the container and the oxygen sensitive product to ionizing radiation at a dosage and energy sufficient to pasteurize the oxygen sensitive product, and trigger the oxygen scavenger of the container.

This application claims the benefit of U.S. Provisional Application No. 60/258,015, filed Dec. 22, 2001.

### FIELD OF THE INVENTION

The invention relates to a process for pasteurizing an oxygen sensitive product and triggering an oxygen scavenger of a container, and the resulting package.

### BACKGROUND OF THE INVENTION

It is known that many oxygen sensitive products, such as food products, benefit from pasteurization or sterilization. Such processes can be accomplished by irradiation from gamma and electron beam sources. Such processes retard microbial, mold, and yeast growth. The radiation processing of food has several advantages over conventional thermal treatments in that it is more energy efficient and can be accomplished in less time.

It is also known that many oxygen sensitive products, including food products, such as meat and cheese, smoked and processed luncheon meats, deteriorate in the presence of oxygen. Both color and flavor can be adversely affected. The oxidation of lipids within the food product can result in the development of rancidity. These products benefit from the use of oxygen scavengers in their packaging. Some of these oxygen scavengers can be triggered or activated by actinic radiation.

It would be desirable to conveniently and simply supply a single packaging material or container, which can be used to package an oxygen sensitive product, and then expose the packaged product and the packaging material itself to actinic radiation sufficiently to pasteurize the contents of the container, and trigger an oxygen scavenger disposed in or on the packaging material or container.

It has now been found that both the challenge of pasteurizing or sterilizing an oxygen sensitive product, and the problem of dealing with oxidative deterioration of an oxygen sensitive product, can be addressed by a single process, which both pasteurizes or sterilizes the oxygen sensitive product, and triggers an oxygen scavenger used in the container for the oxygen sensitive product. The inventors have found that triggering using electron beam (e-beam) radiation, or other forms of actinic or ionizing radiation, is relatively fast. The inventors have also found that an oxygen sensitive product such as a food product, for example, processed meats (bologna, hot dogs, etc.) and ground beef, can be pasteurized and in some cases sterilized, while triggering an oxygen scavenger in or on a container that contains the product. This results in a product with a longer shelf life, and enables oxygen scavenging technology to be integrated into pasteurization and sterilization systems.

Definitions

“Container” herein means an enclosure such as a bag, pouch, or vessel, that is capable of enclosing or packaging an oxygen sensitive product. A container herein can be formed in part by a component such as a tray or lidstock.

“Film” herein means a film, laminate, sheet, web, coating, plastisol, gasket, or the like which can be used to package a product. The film can be used as a component in a rigid, semi-rigid, or flexible product, and can be adhered to a non-polymeric or non-thermoplastic substrate such as paper or metal. A film or sheet can also be used as a coupon or insert within a package.

“Oxygen scavenger”, and the like herein means a composition, compound, film layer, coating, plastisol, gasket, article or the like which can consume, deplete or react with oxygen from a given environment.

“Ionizing radiation” and the like herein means actinic radiation in the form of X-ray, gamma ray, corona discharge, or electron beam irradiation, capable of causing a chemical change, as exemplified in U.S. Pat. No. 5,211,875 (Speer et al.).

“Pasteurized” and the like herein means exposing a material to a treatment process where the material is heated, with radiation, to temperatures and for periods of time sufficient to at least partially pasteurize the material against microbial, mold, and yeast growth, without substantial alteration of the chemical composition of the material. Pasteurized materials are characterized by a prolonged stability against spoilage by microbial and/or mold growth. Thus, the term “pasteurize” is consistent with U.S. Pat. No. 5,474,793 (Meyer et al.), incorporated herein by reference in its entirety, but with the modification that the pasteurization is accomplished with radiation.

The terms “pasteurize” and “pasteurization” include the more restrictive term “sterilize” and the like which refers herein to the effective inactivation or kill of microbes contained in the oxygen sensitive product. The level of inactivation or kill may vary, but it will be in an amount acceptable by the applicable commercial and/or FDA standards for the intended product.

“Polymer” and the like herein means a homopolymer, but also copolymers thereof, including bispolymers, terpolymers, etc.

“Trigger” and the like refers herein to that process defined in U.S. Pat. No. 5,211,875, whereby oxygen scavenging is initiated by exposing a composition, film, etc. to actinic radiation having a wavelength of less than about 750 nm at an intensity of at least about 1.6 mW/cm² or an electron beam at a dose of at least about 0.2 megarads, wherein after initiation the oxygen scavenging rate is at least about 0.05 cc oxygen per day per gram of oxidizable organic compound for at least two days after oxygen scavenging is initiated. Preferred is a method offering a short “induction period” (the time that elapses, after exposing the oxygen scavenger to a source of actinic radiation, before initiation of the oxygen scavenging activity begins) so that the oxygen scavenger can be activated at or immediately prior to use during filling and sealing of the container with an oxygen sensitive material; a method wherein the oxygen scavenging material is substantially consistently triggered across the entire internal surface of the preformed container; a method which is simple and readily incorporated into existing packaging procedures; and a method which is readily incorporated in-line into existing packaging systems.

Thus, “trigger” refers to exposing a composition or article to actinic radiation as described above; “initiation” refers to the point in time at which oxygen scavenging actually begins; and “induction time” refers to the length of time, if any, between triggering and initiation.

### SUMMARY OF THE INVENTION

In a first aspect of the invention, a package comprises a container, the container comprising an oxygen scavenger; and an oxygen sensitive product contained in the container; wherein the oxygen scavenger is triggered, and the oxygen sensitive product is pasteurized.

In a second aspect of the invention, a package comprises a tray; a lidstock in communication with the tray; and an oxygen sensitive product contained in the tray, and enclosed by the lidstock and the tray; wherein at least one of the lidstock and the tray comprises an oxygen scavenger, the oxygen scavenger is triggered, and the oxygen sensitive product is pasteurized.

In a third aspect of the invention, a method comprises providing a container containing an oxygen sensitive product, the container comprising an oxygen scavenger; and exposing the container and the oxygen sensitive product to ionizing radiation at a dosage and energy sufficient to pasteurize the oxygen sensitive product, and trigger the oxygen scavenger of the container.

### DETAILED DESCRIPTION OF THE INVENTION

The Oxygen Scavenger

Oxygen scavengers suitable for commercial use in articles of the present invention, such as films, are disclosed in U.S. Pat. No. 5,350,622, and a method of initiating oxygen scavenging generally is disclosed in U.S. Pat. No 5,211,875. Both applications are incorporated herein by reference in their entirety. According to U.S. Pat. No. 5,350,622, oxygen scavengers are made of an ethylenically unsaturated hydrocarbon and transition metal catalyst. The preferred ethylenically unsaturated hydrocarbon may be either substituted or unsubstituted. As defined herein, an unsubstituted ethylenically unsaturated hydrocarbon is any compound that possesses at least one aliphatic carbon-carbon double bond and comprises 100% by weight carbon and hydrogen. A substituted ethylenically unsaturated hydrocarbon is defined herein as an ethylenically unsaturated hydrocarbon which possesses at least one aliphatic carbon-carbon double bond and comprises about 50%-99% by weight carbon and hydrogen. Preferable substituted or unsubstituted ethylenically unsaturated hydrocarbons are those having two or more ethylenically unsaturated groups per molecule. More preferably, it is a polymeric compound having three or more ethylenically unsaturated groups and a molecular weight equal to or greater than 1,000 weight average molecular weight.

Examples of unsubstituted ethylenically unsaturated hydrocarbons include, but are not limited to, diene polymers such as polyisoprene, (e.g., trans-polyisoprene) and copolymers thereof, cis and trans 1,4-polybutadiene, 1,2-polybutadienes, (which are defined as those polybutadienes possessing greater than or equal to 50% 1,2 microstructure), and copolymers thereof, such as styrene-butadiene copolymer. Such hydrocarbons also include polymeric compounds such as polypentenamer, polyoctenamer, and other polymers prepared by cyclic olefin metathesis; diene oligomers such as squalene; and polymers or copolymers with unsaturation derived from dicyclopentadiene, norbornadiene, 5-ethylidene-2-norbornene, 5-vinyl-2-norbornene, 4-vinylcyclohexene, 1,7-octadiene, or other monomers containing more than one carbon-carbon double bond (conjugated or non-conjugated).

Examples of substituted ethylenically unsaturated hydrocarbons include, but are not limited to, those with oxygen-containing moieties, such as esters, carboxylic acids, aldehydes, ethers, ketones, alcohols, peroxides, and/or hydroperoxides. Specific examples of such hydrocarbons include, but are not limited to, condensation polymers such as polyesters derived from monomers containing carbon-carbon double bonds, and unsaturated fatty acids such as oleic, ricinoleic, dehydrated ricinoleic, and linoleic acids and derivatives thereof, e.g. esters. Such hydrocarbons also include polymers or copolymers derived from (meth)allyl (meth)acrylates. Suitable oxygen scavenging polymers can be made by trans-esterification. Such polymers are disclosed in U.S. Pat. No. 5,859,145 (Ching et al.) (Chevron Research and Technology Company), incorporated herein by reference as if set forth in full. The composition used may also comprise a mixture of two or more of the substituted or unsubstituted ethylenically unsaturated hydrocarbons described above. While a weight average molecular weight of 1,000 or more is preferred, an ethylenically unsaturated hydrocarbon having a lower molecular weight is usable, especially if it is blended with a film-forming polymer or blend of polymers.

Ethylenically unsaturated hydrocarbons which are appropriate for forming solid transparent layers at room temperature are preferred for scavenging oxygen in the packaging articles described above. For most applications where transparency is necessary, a layer which allows at least 50% transmission of visible light is preferred.

When making transparent oxygen-scavenging layers according to this invention, 1,2-polybutadiene is useful at room temperature. For instance, 1,2-polybutadiene can exhibit transparency, mechanical properties and processing characteristics similar to those of polyethylene. In addition, this polymer is found to retain its transparency and mechanical integrity even after most or all of its oxygen uptake capacity has been consumed, and even when little or no diluent resin is present. Even further, 1,2-polybutadiene exhibits a relatively high oxygen uptake capacity and, once it has begun to scavenge, it exhibits a relatively high scavenging rate as well.

When oxygen scavenging at low temperatures is desired, 1,4-polybutadiene, and copolymers of styrene with butadiene, and styrene with isoprene are useful. Such compositions are disclosed in U.S. Pat. No. 5,310,497 issued to Speer et al. on May 10, 1994 and incorporated herein by reference as if set forth in full. In many cases, it may be desirable to blend the aforementioned polymers with a polymer or copolymer of ethylene.

Other oxygen scavengers which can be used in connection with this invention are disclosed in U.S. Pat. No. 5,958,254 (Rooney), incorporated by reference herein in its entirety. These oxygen scavengers include at least one reducible organic compound which is reduced under predetermined conditions, the reduced form of the compound being oxidizable by molecular oxygen, wherein the reduction and/or subsequent oxidation of the organic compound occurs independent of the presence of a transition metal catalyst. The reducible organic compound is preferably a quinone, a photoreducible dye, or a carbonyl compound which has absorbence in the UV spectrum.

An additional example of oxygen scavengers which can be used in connection with this invention are disclosed in PCT patent publication WO 99/48963 (Chevron Chemical et al.), incorporated herein by reference in its entirety. These oxygen scavengers include a polymer or oligomer having at least one cyclohexene group or functionality. These oxygen scavengers include a polymer having a polymeric backbone, cyclic olefinic pendent group, and linking group linking the olefinic pendent group to the polymeric backbone.

An oxygen scavenging composition suitable for use with the invention comprises:
(a) a polymer or lower molecular weight material containing substituted cyclohexene functionality according to the following diagram: 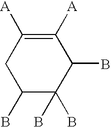
   where A may be hydrogen or methyl and either one or two of the B groups is a heteroatom-containing linkage which attaches the cyclohexene ring to the said material, and wherein the remaining B groups are hydrogen or methyl;
(b) a transition metal catalyst; and optionally
(c) a photoinitiator.

The compositions may be polymeric in nature or they may be lower molecular weight materials. In either case they may be blended with further polymers or other additives. In the case of low molecular weight materials they will most likely be compounded with a carrier resin before use.

When used in forming a packaging article, the oxygen scavenging composition of the present invention can include only the above-described polymers and a transition metal catalyst. However, photoinitiators can be added to further facilitate and control the initiation of oxygen scavenging properties. Adding a photoinitiator or a blend of photoinitiators to the oxygen scavenging composition can be preferred, especially where antioxidants have been added to prevent premature oxidation of the composition during processing and storage.

Suitable photoinitiators are known to those skilled in the art. See, e.g., PCT publication WO 97/07161, WO 97/44364, WO 98/51758, and WO 98/51759 the teachings of which are incorporated herein by reference as if set forth in full. Specific examples of suitable photoinitiators include, but are not limited to, benzophenone, and its derivatives, such as methoxybenzophenone, dimethoxybenzophenone, dimethylbenzophenone, diphenoxybenzophenone, allyloxybenzophenone, diallyloxybenzophenone, dodecyloxybenzophenone, dibenzosuberone, 4,4′-bis(4-isopropylphenoxy)benzophenone, 4-morpholinobenzophenone, 4-aminobenzophenone, tribenzoyl triphenylbenzene, tritoluoyl triphenylbenzene, 4,4′-bis(dimethylamino)benzophenone, acetophenone and its derivatives, such as, o-methoxyacetophenone, 4′-methoxyacetophenone, valerophenone, hexanophenone, α-phenylbutyrophenone, p-morpholinopropiophenone, benzoin and its derivatives, such as, benzoin methyl ether, benzoin butyl ether, benzoin tetrahydropyranyl ether, 4-o-morpholinodeoxybenzoin, substituted and unsubstituted anthraquinones, α-tetralone, acenaphthenequinone, 9-acetylphenanthrene, 2-acetyl-phenanthrene, 10-thioxanthenone, 3-acetyl-phenanthrene, 3-acetylindole, 9-fluorenone, 1-indanone, 1,3,5-triacetylbenzene, thioxanthen-9-one, isopropylthioxanthen-9-one, xanthene-9-one, 7-H-benz[de]anthracen-7-one, 1′-acetonaphthone, 2′-acetonaphthone, acetonaphthone, benz[a]anthracene-7,12-dione, 2,2-dimethoxy-2-phenylacetophenone, α,α-diethoxyacetophenone, α,α-dibutoxyacetophenone, 4-benzoyl-4′-methyl(diphenyl sulfide) and the like. Single oxygen-generating photosensitizers such as Rose Bengal, methylene blue, and tetraphenylporphine as well as polymeric initiators such as poly(ethylene carbon monoxide) and oligo[2-hydroxy-2-methyl-1-[4-(1-methylvinyl)phenyl]propanone] also can be used. However, photoinitiators are preferred because they generally provide faster and more efficient initiation. When actinic radiation is used, photoinitiators also can provide initiation at longer wavelengths, which are less costly to generate and present less harmful side effects than shorter wavelengths.

When a photoinitiator is present, it can enhance and/or facilitate the initiation of oxygen scavenging by the composition of the present invention upon exposure to radiation. The amount of photoinitiator can depend on the amount and type of cyclic unsaturation present in the polymer, the wavelength and intensity of radiation used, the nature and amount of antioxidants used, and the type of photoinitiator used. The amount of photoinitiator also can depend on how the scavenging composition is used. For instance, if a photoinitiator-containing composition is in a film layer, which underneath another layer is somewhat opaque to the radiation used, more initiator might be needed. However, the amount of photoinitiator used for most applications ranges from about 0.01 to about 10% (by wt.) of the total composition. Oxygen scavenging can be initiated by exposing an article containing the composition of the present invention to actinic or electron beam radiation, as described below.

Also suitable for use in the present invention is the oxygen scavenger of co-pending U.S. patent application Ser. No. 09/350,336, filed Jul. 9, 1999, incorporated herein by reference in its entirety, which discloses a copolymer of ethylene and a strained, cyclic alkylene, preferably cyclopentene; and a transition metal catalyst.

Another oxygen scavenger which can be used in connection with this invention is the oxygen scavenger of WO 00/00538, published Jan. 6, 2000, incorporated herein by reference in its entirety, which discloses ethylene/vinyl aralkyl copolymer and a transition metal catalyst.

As indicated above, the ethylenically unsaturated hydrocarbon is combined with a transition metal catalyst. Suitable metal catalysts are those which can readily inter-convert between at least two oxidation states.

Preferably, the catalyst is in the form of a transition metal salt, with the metal selected from the first, second or third transition series of the Periodic Table. Suitable metals include, but are not limited to, manganese II or III, iron II or III, cobalt II or III, nickel II or III, copper I or II, rhodium II, III or IV, and ruthenium II or III. The oxidation state of the metal when introduced is not necessarily that of the active form. The metal is preferably iron, nickel or copper, more preferably manganese and most preferably cobalt. Suitable counterions for the metal include, but are not limited to, chloride, acetate, stearate, palmitate, caprylate, linoleate, tallate, 2-ethylhexanoate, neodecanoate, oleate or naphthenate. Particularly preferable salts include cobalt (II) 2-ethylhexanoate, cobalt stearate, and cobalt (II) neodecanoate. The metal salt may also be an ionomer, in which case a polymeric counterion is employed. Such ionomers are well known in the art.

Any of the above-mentioned oxygen scavengers and transition metal catalyst can be further combined with one or more polymeric diluents, such as thermoplastic polymers which are typically used to form film layers in plastic packaging articles. In the manufacture of certain packaging articles well known thermosets can also be used as the polymeric diluent.

Polymers which can be used as the diluent include, but are not limited to, polyethylene terephthalate (PET), polyethylene, low or very low density polyethylene, ultra-low density polyethylene, linear low density polyethylene, polypropylene, polyvinyl chloride, polystyrene, and ethylene copolymers such as ethylene-vinyl acetate, ethylenealkyl(meth)acrylates, ethylene-(meth)acrylic acid and ethylene-(meth)acrylic acid ionomers. Blends of different diluents may also be used. However, as indicated above, the selection of the polymeric diluent largely depends on the article to be manufactured and the end use. Such selection factors are well known in the art.

Further additives can also be included in the composition to impart properties desired for the particular article being manufactured. Such additives include, but are not necessarily limited to, fillers, pigments, dyestuffs, antioxidants, stabilizers, processing aids, plasticizers, fire retardants, anti-fog agents, etc.

The mixing of the components listed above is preferably accomplished by melt blending at a temperature in the range of 50° C. to 300° C. However, alternatives such as the use of a solvent followed by evaporation may also be employed. The blending may immediately precede the formation of the finished article or preform or precede the formation of a feedstock or masterbatch for later use in the production of finished packaging articles.

Oxygen scavenging structures can sometimes generate reaction byproducts, which can affect the taste and smell of the packaged material (i.e. organoleptic properties), or raise food regulatory issues. These by-products can include organic acids, aldehydes, ketones, and the like. This problem can be minimized by the use of polymeric functional barriers. Polymeric functional barriers for oxygen scavenging applications are disclosed in WO 96/08371 to Ching et al. (Chevron Chemical Company), WO 94/06626 to Balloni et al., and copending U.S. patent application Ser. No. 08/813752 (Blinka et al.) and Ser. No. 09/445645 (Miranda), all of which are incorporated herein by reference as if set forth in full. The materials in these publications and applications collectively include high glass transition temperature (T_{g}) glassy polymers such as polyethylene terephthalate (PET) and nylon 6 that are preferably further oriented; low T_{g} polymers and their blends; a polymer derived from a propylene monomer; a polymer derived from a methyl acrylate monomer; a polymer derived from a butyl acrylate monomer; a polymer derived from a methacrylic acid monomer; polyethylene terephthalate glycol (PETG); amorphous nylon; ionomer; a polymeric blend including a polyterpene; and poly (lactic acid). The functional barrier polymer(s) may further be blended with another polymer to modify the oxygen permeability as required by some applications. The functional barriers can be incorporated into one or more layers of a multilayer film, container, or other article that includes an oxygen scavenging layer. In certain applications of oxygen scavenging, it is desirable to provide polymeric materials with low oxygen transmission rates, i.e. with high barrier to oxygen. In these cases, it is preferred that the oxygen permeability of the barrier be less than 500 cm^{3 O}₂/m²·day·atmosphere (tested at 1 mil thick and at 25° C. according to ASTM D3985), preferably less than 1 00, more preferably less than 50 and most preferably less than 25 cm³ O₂/m²·day·atmosphere such as less than 10, less than 5, and less than 1 cm³ O₂/m²·day·atmosphere. Examples of polymeric materials with low oxygen transmission rates are ethylene/vinyl alcohol copolymer (EVOH), polyvinylidene dichloride, vinylidene chloride/methyl acrylate copolymer, polyamide, polyester; and metallized PET. Alternatively, metal foil or SiOx compounds can be used to provide low oxygen transmission to the container. The exact oxygen permeability optimally required for a given application can readily be determined through experimentation by one skilled in the art. In medical applications, high barrier is often required to protect the quality of the product being packaged over the intended lifetime of the product. Higher oxygen permeability can readily be accomplished by blending the barrier polymer with any polymer that has a substantially higher oxygen permeability. Useful polymers for blending with barrier polymers include but are not limited to polymers and copolymers of alkyl acrylates, especially ethylene/butyl acryate, ethylene/vinyl acetate copolymers, and the like.

The Ionizing Radiation

Ionizing radiation will penetrate materials to a given depth that depends on the density of the material, the atomic number of the material, and the energy of the radiation. In the case of an electron beam, the energy is determined by the acceleration voltage of the e-beam apparatus and is frequently measured in kilo or mega volts (kV or MV). The energy of the ionizing radiation is measured in kilo or mega electron volts (keV or MeV) and is attenuated by increasing distance from the source. The energy is also attenuated to an increasing extent by materials that have greater atomic numbers. Materials containing elements with atomic numbers greater than that of carbon and hydrogen will, for example, attenuate the radiation more than a hydrocarbon polymer for a given thickness. E-beams used with this invention will typically be operated at accelerating voltages of greater than 200,000 electron volts depending upon the product being irradiated.

Suitable gamma irradiation sources include radioisotopes such as cobalt-60 or cesium-137. The energy of gamma rays given off by cobalt-60 is about 1.25 MeV, while cesium-137 is about half that value.

The dose of ionizing radiation is measured in terms of the quantity of energy absorbed per unit mass of irradiated material; units of measure in general use are the megarad (Mrad) and kiloGray (kGy). The dose required to treat a product is highly variable and depends upon the product being irradiated and the microorganisms being controlled. In some cases, a dose as low as 0.1 kGy may be effective, while in other cases, a dose of 40 to 50 kGy may be required for the desired level of control. Thus, a dose of ionizing radiation in connection with the invention can be at least 0.1 kGy, such as at least 0.5 kGy, or at least 1, 5, 10, or 20 kGy. A dose of ionizing radiation in connection with the invention can be between 1 and 50 kGy, such as between 10 and 40, between 20 and 30, or between 40 and 50 kGy.

### EXAMPLES

### Example 1

A first set of pouches (Set 1) were made from an experimental film containing an oxygen scavenger, poly(ethylene/methyl acrylate/cyclohexene-methyl acrylate or EMCM, with a 0.5 mil thick sealant.

A second set of pouches (Set 2) were made from a commercial film, R660B, available from Cryovac, Inc.

The R660B film has the following structure:

| | | | | |
|---|---|---|---|---|
| PVDC-coated | Adhesive | LLDPE | 85% LLDPE + 15% | LLDPE |
| PET film | | | LDPE | |

Both sets of pouches were filled with various levels of oxygen and received an electronic irradiation dose of 5 kGy. These pouches were tested for scavenging activity to determine if the irradiation would trigger the oxygen scavenging reaction. None of the irradiated pouches exhibited any immediate scavenging activity, but some did exhibit scavenging activity after a period of time. The pouches were filled with bologna, vacuum packaged, and irradiated at a dose of 5 kGy. The color of the bologna was monitored over time. Color data was inconclusive; however, bologna in the Set 2 pouches developed patches of discoloration after day 43. Bologna in the Set 1 pouches made from the oxygen scavenger film never developed discoloration during the day test. On day 65, the bologna pouches were evaluated for scavenging activity. The level of headspace oxygen in the Set 1 pouches was significantly reduced compared to the level in the Set 2 pouches, indicating that the oxygen scavenger in the Set 1 pouches was indeed scavenging oxygen. It is possible that the display case lights, where the bologna packages were stored, contributed to triggering of the oxygen scavenging reaction in the Set 1 pouches due to the presence of some UV light in the display case lights. This test indicates that the scavenging packages successfully preserved the color of the bologna.

### Comparative Example 1

Using the Set 1 and Set 2 materials, 10″×10″ (200 in² area) pouches were made. Both sets contained the same saran coated polyethylene terephthalate as the physical oxygen barrier. In order to evacuate atmospheric gases from the pouches, each pouch was vacuumized and sealed in a Koch Ultravac™ 250. Three pouches of each material were filled with approximately 600 cc of 20.6% O₂, and three pouches of each material were filled with 1% O₂. Headspace O₂ analysis was conducted on these packages after they were filled to determine the initial headspace concentration. Five days after these pouches as well as vacuumized pouches were produced in the lab they were irradiated using electronic beam with a beam energy of 10 million electron volts (MeV). Each package received a dose of 5 kGy.

One day after irradiation, headspace analysis was conducted on packages filled with 20.6% and 1% O₂. Seven days after irradiation two each of Set 1 pouches filled with 20.6 or 1% O₂ were opened, and the film was activated using a UV radiation apparatus. Each sealant side of the pouch (inside of pouch) was dosed with 1600 mJ/cm² of UVC light. Film was sealed back into pouches and vacuumized. These pouches were again filled with approximately 600 cc of 20.6% and 1% O₂. Headspace O₂ analysis was conducted on day 0 and 1.

Eight days after irradiation three of the packages from each film that remained vacuumized during irradiation were filled with 1% O₂. Headspace O₂ analysis was conducted on day 0 and day 6.

### Example 2

Bologna Study

Five pouches of each material were each filled with 4 thick bologna slices and each pouch was vacuumized. Five days after the pouches were made in the lab they were irradiated using an electron beam with at 5 MeV. Packages received a 5 kGy dose. One day after irradiation, the packages containing bologna were placed in a display case. Color of bologna was determined using a Minolta Colorimeter, and the Hunter L, a, b scale.

Pouches made from Set 1 and Set 2 were irradiated 5 days after they were filled with 20.6 and 1% O₂. By day 7 (two days after irradiation), there was no decrease in O₂ levels as would be expected in the Set 1 pouches if the oxygen scavenger in the pouch material had been activated by the electron beam (Table 1).

TABLE 1
Headspace O₂ in pouches that contained
600 cc of 20.6 or 1% O₂ during irradiation.

| | % O₂ | | |
|---|---|---|---|
| Film | Day 0 | Day 6* | Day 7 |
| Set 2 | 21.0 | 20.7 | 20.6 |
| Set 1 | 21.1 | 20.6 | 20.7 |
| Set 2 | 1.10 | 1.11 | 1.14 |
| Set 1 | 1.08 | 1.12 | 1.14 |

*Packages irradiated on day 5.

Irradiated vacuumized pouches were filled with 1% O₂ eight days after irradiation to determine if the electron beam activated the oxygen scavenging reaction under minimal oxygen levels. Oxygen scavenger film is more easily activated in the presence of oxygen because oxygen helps to set off the oxygen scavenging reaction. Often the introduction of O₂ will increase the scavenging rate of a film that has been activated. When these vacuum packaged pouches were filled with 1% O₂, the O₂ level's steady increase suggested the oxygen scavenging reaction was not activated by this particular irradiation process(see Table 2).

TABLE 2
Headspace O₂ of packages that remained vacuumized
during irradiation and then filled with 1% O₂ 8 days later.

| | % O₂ | | |
|---|---|---|---|
| Film | Day 0* | Day 1 | Day 8 |
| Set 1 | 0.983 | 1.06 | 1.23 |
| Set 2 | 0.983 | 1.06 | 1.28 |

*Packages filled with O₂ on day 0 (eight days after irradiation treatment).

In order to determine if the Set 1 pouches could not be activated, irradiated Set 1 pouches were opened up and exposed to 1600 mJ/cm² of UV light. Such an exposure to UV light is known to activate oxygen scavenger film. Pouches were filled with 20.6% or 1% O₂ following exposure to UVC light. By day 1, the O₂ levels in the pouches had been reduced significantly (Table 3). These results suggested that the film was triggered with 1600 mJ/cm², but was not immediately triggered by the irradiation process. Other studies indicate that the induction time may exceed the duration of the particular study. This is especially so when very low oxygen levels are initially present and/or when films are used at subambient temperature conditions.

TABLE 3
Headspace O₂ in irradiated packages that
were dosed with 1600 mJ/cm² of UVC
light eight days after initial irradiation.

| | % O₂ | | |
|---|---|---|---|
| Film | Day 0* | Day 1 | Day 8 |
| Set 1 | 20.9 | 7.605 | 4.465 |
| Set 1 | 0.983 | 0.0936 | 0.0339 |

*Packages dosed with 1600 mJ/cm² of UVC light on day 0 (eight days after irradiation treatment).

### Example 3

Bologna Study

Color of the bologna packages changed during the 99-day tests (Table 4 and 5). However, the color of the film also changed during the 99-day test. Set 1 pouches tended to yellow over time. By day 30 a yellowish tint was detectable by the human eye on the Set 1 pouches. Therefore, calorimeter readings of both films were taken at each sample period after day 30. To do this, a clear section of each pouch was placed against the white calibration tile, and calorimeter readings were recorded (Table 6). Calibration tile calorimeter readings are Hunter L=96.69, a=−0.07, and b=1.93. Although the difference in the calibration tile and film color could be subtracted from the package calorimeter readings to give a true bologna color, this is not the color consumers would see when buying this product. During this study, the light bulbs in the display case where the bologna was stored were changed. Between day 48 and 52, Cool White™ 40-watt bulbs were replaced with GE RE830™ bulbs.

TABLE 4
Average Hunter L, a, b colorimeter reading for bologna vacuum
packaged in Set 2 (non-scavenging) irradiated pouches.

| Day | L | A | B |
|---|---|---|---|
| 9 | 62.59 a¹ | 8.88 a | 13.69 a |
| 22 | 62.61 a | 12.44 b, d | 10.77 b |
| 30 | 62.60 a | 13.01 c | 10.89 c |
| 43 | 61.88 b, c | 13.31 c | 10.85 b, c |
| 59 | 61.71 b, c, d | 12.31 d, e | 11.26 d |
| 65 | 62.12 c, d | 12.30 b, e | 11.37 d |
| 99 | 61.37 d | 12.32 b, e | 11.52 d |

¹Values followed by the same letter within the same column are not significantly different (p ≦ 0.05) as determined by least square means.

TABLE 5
Average Hunter L, a, b colorimeter readings for bologna vacuum
packaged in Set 1 (scavenging) pouches irradiated.

| Day | L | a | b |
|---|---|---|---|
| 9 | 62.51 a¹ | 9.51 a | 13.84 a |
| 22 | 63.19 b | 13.05 b | 10.82 b |
| 30 | 62.22 a | 12.47 c | 10.87 b, c, d |
| 43 | 62.46 a | 13.89 d | 10.69 b |
| 59 | 62.97 b | 13.01 b | 11.18 c, d |
| 65 | 62.94 b | 12.88 b, e | 11.00 b, c |
| 99 | 63.32 b | 12.50 c, e | 11.31 d |

¹Values followed by the same letter within the same column are not significantly different (p ≦ 0.05) as determined by least square means.

TABLE 6
Average Hunter L, a, b colorimeter readings for Set 2
(non-scavenging) film e-beam irradiated.

| Day | L | a | B |
|---|---|---|---|
| 30 | 93.52 | −0.08 | 3.04 |
| 59 | 93.97 | −0.23 | 3.50 |
| 65 | 93.33 | −0.24 | 3.78 |
| 99 | 92.96 | −0.38 | 4.64 |

TABLE 7
Average Hunter L, a, b colorimeter readings for Set 1
(scavenging) film e-beam irradiated.

| Day | L | a | B |
|---|---|---|---|
| 30 | 93.08 | −0.23 | 3.70 |
| 59 | 93.19 | −0.44 | 4.33 |
| 65 | 92.93 | −0.51 | 4.70 |
| 99 | 92.27 | −0.73 | 5.56 |

The data in Tables 6 & 7 indicate that both films yellow to some extent upon exposure to e-beam irradiation.

Although the calorimeter results do not indicate this, on day 43 discoloration was observed on bologna packaged in the non-scavenging Set 2 pouches. Greenish white areas appeared on the surface of the bologna and in creases created by vacuum packaging. These spots continued to increase during the 99-day study in the non-scavenging Set 2 pouches. No discoloration was observed during the 99-day study on the bologna packaged in the scavenging Set 1 pouches. Both light and oxygen must be present for the greenish white discoloration to occur on bologna. To verify that the Set 1 pouches were indeed scavenging oxygen, one bologna package of each film was filled with 600 cc of 1% O₂ and left at room temperature for 7 days. This was carried out on day 65. Results indicated that indeed the Set 1 pouches were scavenging O₂ (Table 7).

On day 7, total aerobes and Tactics were enumerated. Total aerobes and lactics were >6.0 Log CFU/g [need to explain units] on bologna stored in the non-scavenging Set 2 pouches. These microbial counts explain the low residual headspace oxygen (Table 8) seen in non-scavenging Set 2 pouches. In this pouch, aerobic bacteria were utilizing the residual headspace oxygen thereby reducing it by 10%.

TABLE 8
Percent headspace O₂ in a Set 2 and Set 1
pouches 65 days after electron beam irradiation.

| | % O₂ | | |
|---|---|---|---|
| Film | Day 0 | Day 4 | Day 7 |
| Set 1 | 1.08 | 0.0639 | 0.0596 |
| Set 2 | 1.06 | 0.759 | 0.114 |

TABLE 9
Total aerobes and lactics on bologna in (scavenging)
Set 1 and (control) Set 2.

| | Aerobes | Lactics |
|---|---|---|
| Film | (Log CFU/g) | (Log CFU/g) |
| Set 1 | 4.56 | 4.52 |
| Set 2 | >6.00 | >6.00 |

In this study, it was uncertain whether an irradiation dose of 5 kGy at 10 MeV activated the oxygen scavenging reaction in the Set 1 pouches. It is believed that a lower energy beam would be more effective in activating the film without an induction period. These results indicate that packaging and radiation processing bologna in an oxygen scavenging film extends the shelf life as compared to radiation processing alone.

Electron beam exposure to 5 kGy with 10 MeV may not have triggered the oxygen scavenger film so as to achieve an induction time of less than 1 day. The effect on induction is complicated by the very low initial volumes of oxygen and low temperature storage conditions. It has been shown that exposure to a 7 KeV with a 3 kGy dose did induce oxidation when 2% oxygen (volume percent) at 23° C. conditions were used. Regardless, the e-beam-exposed oxygen scavenger film did prolong the shelf life of the e-beam pasteurized bologna as evidenced by the difference in aerobics and lactics bacteria counts.

The invention can be used in connection with various articles of manufacture, compounds, compositions of matter, coatings, etc. Two preferred forms are sealing compounds, and flexible films, both useful in packaging of food and non-food products. In addition to caps and closures, and traditional flexible film applications, the invention can be used in association with semirigid packaging, rigid containers, foamed and unfoamed trays, and paperboard liners, in systems where an oxygen scavenger has been triggered.

It is known to use sealing compounds in the manufacture of gaskets for the rigid container market. Large, wide diameter gaskets are typically made using a liquid plastisol. This plastisol is a highly viscous, liquid suspension of polymer particles in a plasticizer. In the manufacture of metal or plastic caps, lids, and the like, this liquid plastisol is applied to the annulus of a container such as a jar, and the container with the applied plastisol is “fluxed” in an oven to solidify the plastisol into a gasket. The result is a gasket formed around the annulus of the container.

Smaller gaskets are typically made for use in beer crowns in bottles. A polymer melt is applied by cold molding to the entire inner surface of the crown. Both poly(vinyl chloride) (PVC) and other polymers are used in this application.

Discs for plastic caps are typically made by taking a ribbon of gasket material and making discs, and inserting the discs into the plastic cap.

The invention can be used in the packaging of a wide variety of oxygen sensitive products including fresh red meat such as beef, pork, lamb, and veal, smoked and processed meats such as sliced turkey, pepperoni, ham and bologna, vegetable products such as tomato based products, other food products, including pasta and baby food, beverages such as beer, and products such as electronic components, pharmaceuticals, medical products, and the like. The invention is readily adaptable to various vertical form-fill-and-seal (VFFS) and horizontal form-fill-and-seal (HFFS) packaging lines.

Two specific package configurations with which the present invention can be used are a modified atmosphere (MAP) package and a vacuum package. In a MAP package, a food product such as meat or cheese is placed on a solid or foamed tray or thermoformed pouch, and then covered by a lidstock in a conventional manner. At some time during the packaging process, the interior environment of the package is flushed with a gas such as carbon dioxide, nitrogen, or some combination thereof, to replace the air inside the package. The tray, the lidstock, or both can include an oxygen scavenger. In a vacuum package, a food product such as meat or cheese is placed on a solid or foamed tray, sheet, or bottom web, and then covered by a lidstock in a conventional manner. At some time during the packaging process, a vacuum is drawn on the interior environment of the package to remove air from the interior of the package. Both the tray and the lidstock can include an oxygen scavenger.

The invention is not limited to the illustrations described herein, which are deemed to be merely illustrative, and susceptible of modification of form, size, arrangement of parts and details of operation.

1-12. (canceled)

13. A method comprising:
a) providing a container containing an oxygen sensitive product, the container comprising an oxygen scavenger; and
b) exposing the container and the oxygen sensitive product in a single process to ionizing radiation at a dosage and energy sufficient to
i) pasteurize the oxygen sensitive product, while
ii) triggering the oxygen scavenger of the container.

14. The method of claim 13 comprising providing a container made from a film.

15. The method of claim 13 comprising providing a container made from a film comprising:
a) a first layer comprising an oxygen scavenger; and
b) a second layer comprising a polymer having an oxygen transmission rate of less than 500 cm³/m²·day·atm (ASTM D 3985-95).

16. The method of claim 15 wherein the polymer having an oxygen transmission rate of less than 500 cm³/m²·day·atm comprises a polymer selected from the group consisting of:
i) ethylene/vinyl alcohol copolymer,
ii) polyvinylidene dichloride,
iii) vinylidene chloride/methyl acrylate copolymer,
iv) polyamide,
v) polyester; and
vi) metallized PET.

17. The method of claim 13 comprising providing a container containing an oxygen sensitive product, the container comprising an oxygen scavenger, wherein the oxygen scavenger comprises a material selected from the group consisting of:
i) oxidizable organic compound and a transition metal catalyst,
ii) ethylenically unsaturated hydrocarbon and a transition metal catalyst,
iii) a reduced form of a quinone, a photoreducible dye, or a carbonyl compound which has absorbence in the UV spectrum,
iv) a polymer having a polymeric backbone, cyclic olefinic pendent group, and linking group linking the olefinic pendent group to the polymeric backbone,
v) a copolymer of ethylene and a strained, cyclic alkylene, and
vi) ethylene/vinyl aralkyl copolymer.

18. The method of claim 13 wherein the container and the oxygen sensitive product are exposed to ionizing radiation at a dosage of at least 0.1 kGy.

19. The method of claim 13 wherein the container and the oxygen sensitive product are exposed to ionizing radiation at a dosage of between 1 kGy and 50 kGy.

20. The method of claim 13 wherein the oxygen sensitive product is a food product.
